(19) United States
(12) Patent Application Publication
(10) Pub. No.: US 2008/0287788 A1
(43) Pub. Date: Nov. 20, 2008

(54) SYSTEMS AND METHODS FOR ORGAN MONITORING

(75) Inventors: Charles L. Richardson, Monroe, NC (US); Michael L. Smith, Matthews, NC (US); Franz W. Kellar, Gastonia, NC (US)

Correspondence Address: Altera Law Group, LLC, 220 S 6 St Suite 1700, Minneapolis, MN, 55402, US

(73) Assignee: LIFESCIENCE SOLUTIONS, LLC, Charlotte, NC (US)

(21) Appl. No.: 11/748,425

(22) Filed: May 14, 2007

(51) Int. Cl. A61B 8/06

(52) U.S. Cl. 600/437; 600/504; 600/547; 705/3

(57) ABSTRACT

A method for monitoring a patient's organ includes: inputting an electrical signal into the organ; receiving the electrical signal from the organ; and comparing the received electrical signal to a reference electrical signal to determine whether the patient's organ is functioning properly. The electrical signals may be representative of flow characteristics. In one aspect, a system for monitoring a patient's organ includes a sensor sock having a flexible body adapted to at least partially surround an organ, the sock carrying a plurality of spaced-apart electrodes. In another aspect, the system includes at least one flow transducer adapted to be attached to a blood vessel connected to the organ. A sensor unit is adapted to be implanted into the patient's body and to transmit and receive electrical signals from the electrodes or transducers. A computer may be programmed to compare the received electrical signal to the reference electrical signal.

### BACKGROUND OF THE INVENTION

The present invention relates generally to systems and methods for monitoring and evaluating organ function and, more particularly, to non-invasive apparatus and methods for monitoring and evaluating the function of transplanted organs, detecting organ failure, and providing an appropriate warning to the patient and/or physician in the event of actual or anticipated organ failure.

Organ failure, particularly kidney failure, can be disabling or fatal. Renal failure is often treated by dialysis. This provides a substitute for the kidney function, but there are disadvantages associated with it, including the time, expense, and lifestyle changes required. Accordingly, when a donated kidney is available, a transplant (allograft) from a living or cadaver donor is performed on the patient. Each year approximately 14,000 kidney transplants are performed in the United States alone. The average one-year survival rate is about 95%. The most common cause of death is infection, followed by acute rejection. The currently available apparatus and methods for monitoring a transplanted kidney or for assisting in kidney failure assessment are quite limited and, for the most part, require the patient to undergo extensive invasive procedures or repetitive visits to a hospital or other medical facility which can be expensive. Furthermore, such methods are not usually effective in identifying incipient rejection at an early stage.

Known methods for monitoring patients who receive a kidney or other organ transplant typically require an invasive biopsy of the organ. The patient is taken to a laboratory and one or more small pieces of the organ are sampled, which are then sent for pathological evaluation. This procedure is expensive, invasive, and can not identify incipient organ failure which begins in localized regions of the organ. Furthermore, in an already immune-compromised patient, the biopsy procedure itself may cause damage which could precipitate organ failure.

Medical practitioners have attempted to reduce the risks associated with biopsies by exploring alternative methods for predicting transplant rejection. For example, a method of rejection monitoring is disclosed in U.S. Pat. No. 5,246,008 to Mueller. As disclosed in Mueller, a rejection monitor (“RM”) is connected to a patient's organ using current and measuring electrodes in which each current electrode is annularly surrounded by a measuring electrode. This RM includes a miniaturized, battery-operated electronic measuring circuit for impedance measurement and a transmitter-receiver. An AC voltage is applied in a square-wave pulse to the tissue via the current electrodes. The impedance of the body tissue is then measured via the measuring electrodes.

As disclosed in Mueller, the impedance consists substantially of the ohmic resistance and a capacitive reactance. The ohmic resistance depends substantially on the extracellular space of the tissue, whereas the capacitive reactance depends substantially on the properties of the cell membrane. As a result of ischemia of the tissue during a rejection reaction, intracellular edema with simultaneous shrinkage of the extracellular space occurs, which results in changes to the ohmic resistance and capacitive reactance of the tissue. The change of the pulse form of the ac voltage is a measure of the impedance. If a square-pulse voltage is used as the ac voltage, the change of the pulse height corresponds to the ohmic resistance, whereas the change in the steepness of the leading edges of the square-wave pulses is a measure of the capacitive reactance.

While Mueller provides an alternative to invasive biopsy, the system and method described therein is not believed to be sensitive to incipient cell degradation which may begin at locations remote from the electrodes. Furthermore, Mueller requires individual placement of the electrodes on the surface of the organ.

### BRIEF SUMMARY OF THE INVENTION

These and other shortcomings in the prior art are addressed by the present invention, which according to one aspect provides a method for monitoring a patient's organ, including: (a) inputting an electrical signal into the organ at a first location; (b) receiving the electrical signal from the organ at a second location spaced-apart from the first location; and (c) comparing the received electrical signal to a reference electrical signal to determine whether the patient's organ is functioning properly.

According to another aspect of the invention, a method for monitoring a patient's organ includes: (a) measuring a first flow characteristic within a blood vessel which is connected to the organ; and (b) comparing the first flow characteristic to a reference flow characteristic to determine whether the patient's organ is functioning properly.

According to another aspect of the invention, a system for monitoring a patient's organ includes: (a) A sensor sock comprising a flexible body adapted to at least partially surround an organ, the sock carrying a plurality of spaced-apart electrodes; and (b) a sensor unit adapted to be implanted into the patient's body, the sensor unit connected to the electrodes and adapted to transmit and receive electrical signals from the electrodes.

According to another aspect of the invention, a system for monitoring a patient's organ includes: (a) At least one transducer adapted to be attached to a blood vessel connected to the organ, and to sense at least one characteristic of flow inside the blood vessel; and (b) a sensor unit adapted to be implanted into the patient's body, the sensor unit connected to the transducer and adapted to transmit and receive electrical signals from the transducer.

According to another aspect of the invention, a system for monitoring a patient's organ includes: (a) a sensor unit adapted to be implanted into the patient's body, and to register an electrical signal from the patient's organ; and (b) a local data unit in operable communication with the sensor unit, the local data unit configured to receive and store data from the sensor unit and to selectively transmit the received data over a communications path.

According to another aspect of the invention, a method of monitoring a transplanted organ includes: (a) during a first data collection session occurring at a reference time, injecting a predetermined electrical signal into a patient's organ; (b) during the first data collection session, registering a resulting electrical signal from the organ, the resulting electrical signal configured as a first series of waveforms; (c) generating from the first series of waveforms, a reference waveform representative of the average characteristics of the waveforms collected during the first data collection session; (d) during a subsequent data collection session occurring at a time subsequent to the reference time, injecting the predetermined electrical signal into the patient's organ; (e) during the subsequent data collection session, registering a resulting electrical signal from the organ, the electrical signal configured as a second series of waveforms; (f) generating from the second series of waveforms, a registered waveform representative of the average characteristics of the waveforms collected during the subsequent data collection session; and (g) comparing the registered waveform to the reference waveform to determine whether the organ is functioning properly.

According to another aspect of the invention, a method of monitoring a transplanted organ includes: (a) during a data collection session, injecting a predetermined electrical signal into a patient's organ, the electrical signal configured as a series of waveforms; (b) during the data collection session, registering a resulting electrical signal from a patient's organ, the electrical signal configured as a series of waveforms; (c) evaluating whether each of the waveforms is usable according to a predetermined standard; (d) discarding waveforms which are not usable; (e) storing the remaining waveforms in a database for evaluation; and (f) comparing the stored waveforms to a reference waveform to determine whether the organ is functioning properly.

According to another aspect of the invention, a method of processing data for monitoring a patient's organ includes: (a) during a data collection session, injecting an electrical signal into a patient's organ; (b) during the data collection session, registering a resulting electrical signal from the organ, the electrical signal configured as a series of waveforms, wherein each of said waveform includes at least one upslope element extending to a peak; (c) establishing a minimum slope value; (d) comparing the actual slope value of each portion of the upslope to the minimum slope value; and (e) designating any point within the waveform in which the actual slope value is less than the minimum slope value to be a peak.

### BRIEF DESCRIPTION OF THE DRAWINGS

The invention may be best understood by reference to the following description taken in conjunction with the accompanying drawing figures in which:

FIG. 1 is a schematic cross-sectional view of a kidney, illustrating some of its internal structures;

FIG. 2 is a schematic side view of a kidney connected to a sensor unit constructed according an aspect of the present invention;

FIG. 3 is a cross-sectional view of an implantable sensor unit constructed in accordance with an aspect of the present invention;

FIGS. 4A and 4B are end and side views, respectively, of a first variation of a sensor sock constructed according to the present invention;

FIG. 5 is an interior view of a portion of the sensor sock shown in FIGS. 4A and 4B, showing an electrode thereof;

FIG. 6 is a cross-sectional view taken along lines 6-6 of FIG. 5;

FIG. 7 is a schematic side view of the sensor sock of FIGS. 4A and 4B disposed around a kidney;

FIGS. 8A and 8B are end and side views, respectively, of another variation of a sensor sock constructed according to the present invention;

FIG. 9 is a schematic side view of the sensor sock of FIGS. 8A and 8B disposed around a kidney;

FIGS. 10A and 10B are end and side views, respectively, of another variation of a sensor sock constructed according to the present invention;

FIG. 11 is a schematic side view of the sensor sock of FIGS. 10A and 10B disposed around a kidney;

FIG. 12 is a diagram showing a digitized waveform or waveform, according to one aspect of the present invention;

FIG. 13 is a diagram illustrating an exemplary waveform with a hysteresis band applied thereto;

FIG. 14 is a block diagram showing a data processing flow in accordance with an aspect of the present invention;

FIG. 15 is a diagram illustrating the measurement of the total area under the peak of a waveform;

FIG. 16 is a diagram the measurement of the baseline-to-peak amplitude of a waveform;

FIG. 17 is a diagram illustrating the measurement of the total duration of a waveform;

FIG. 18 is a diagram illustrating the measurement of the slope of the leading edge of a waveform;

FIG. 19 is a diagram graphically illustrating an area comparison of a first waveform, which corresponds to a registered electrical signal from a patient's kidney, to a second waveform, which corresponds to a reference electrical signal from the patient's kidney, according to one aspect of the present invention;

FIG. 20 is a diagram graphically illustrating a point comparison of a first waveform, which corresponds to a registered electrical signal from a patient's kidney, to a second waveform, which corresponds to a reference electrical signal from the patient's kidney, according to one aspect of the present invention;

FIG. 21 is a schematic side view of a kidney having a plurality of flow sensors attached thereto in a first arrangement;

FIG. 22 is a schematic side view of a kidney having a plurality of flow sensors attached thereto in an alternative arrangement;

FIG. 23 is a schematic side view of a kidney having a flow sensor attached thereto in another alternative arrangement;

FIG. 24 is a block diagram showing a portion of a data collection system for use with the present invention; and

FIG. 25 is block diagram showing another portion of the data collection system of FIG. 24.

### DETAILED DESCRIPTION OF THE INVENTION

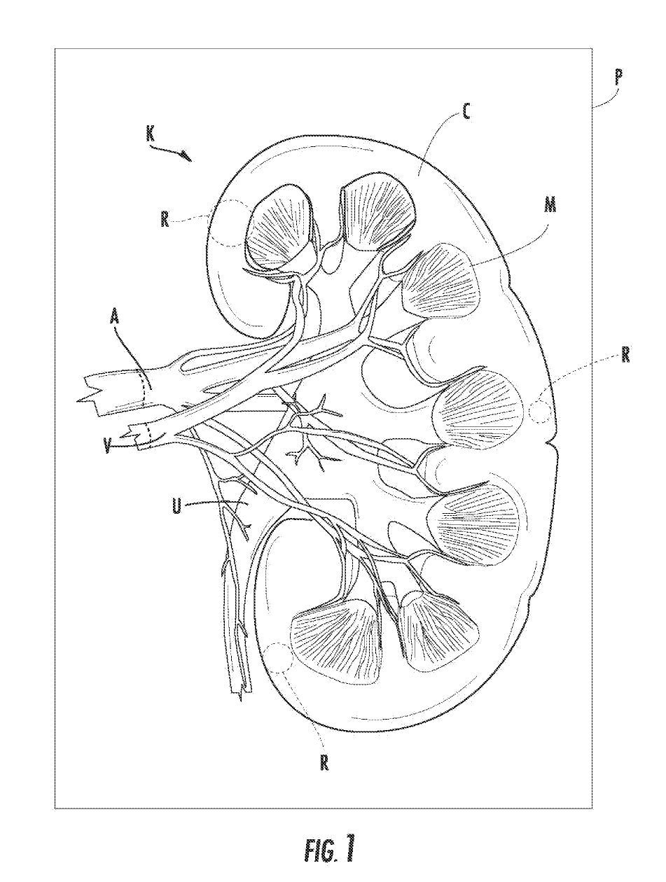

Referring to the drawings wherein identical reference numerals denote the same elements throughout the various views, FIG. 1 illustrates a transplanted kidney “K” in a patient “P” showing some of its structures including renal vein “V”, renal artery “A”, ureter “U”, medulla “M”, and cortex “C”. The kidney K is shown merely as an example, and the systems and methods described herein may be used with other organs. In normal functioning, blood is pumped into the kidney K through renal artery A. The kidney K processes the blood in a known manner, and wastes and excess water are discharged through the ureter U. The cleaned blood is returned to the body through renal vein V. When rejection takes place after a transplant, cell degradation is generally believed to begin first in the cortex C. FIG. 1 illustrates several localized regions “R” of incipient cell degradation. Initially, these regions R encompass a small percentage of the volume of the kidney K, later growing to encompass a majority or all of the kidney K. Accordingly, if rejection is chronic, a conventional biopsy will have a low probably of extracting tissue from one of these regions R. This will have the effect of either delaying diagnosis until the rejection is advanced, or requiring frequent biopsies (which are undesirable for the reasons of cost, risk, and patient discomfort described above). Furthermore, conventional methods of non-invasive evaluation, such as the impedance measurement technique described in Mueller, using closely-spaced sending and receiving electrodes, may not be sufficiently sensitive to determine the presence of the localized cell degradation, because these local regions R may be too remote from any surface electrode to cause a meaningful change in the impedance value.

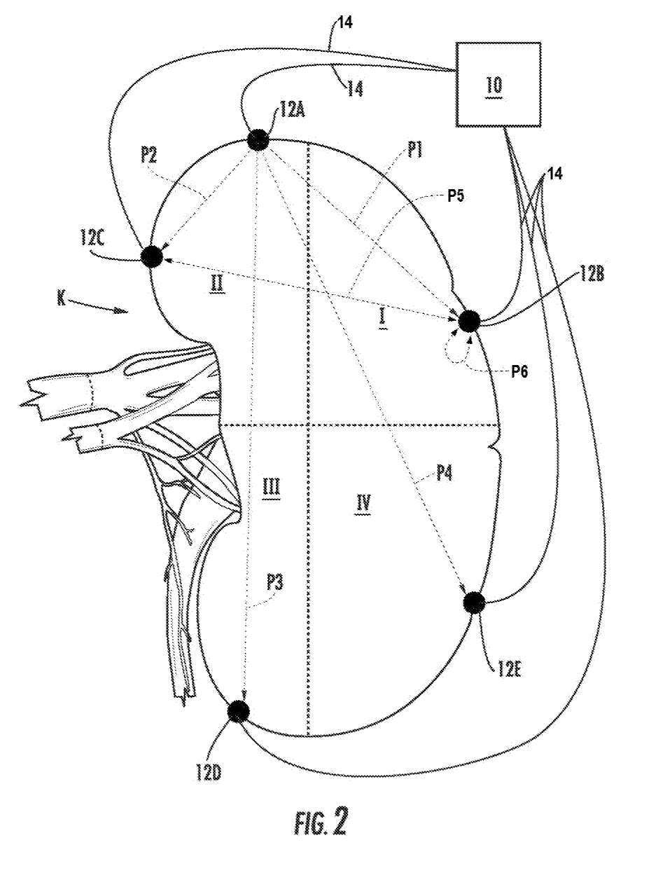

FIG. 2 illustrates a kidney K connected to an implanted sensor unit 10 constructed according to an aspect of the present invention. One or more electrodes 12 contact the kidney K and are coupled to the sensor unit 10 through leads 14. These electrodes 12 may be of a unipolar configuration or multipolar configuration (e.g. bipolar, tripolar). If multipolar electrodes are provided, they may be used in either unipolar or multipolar mode. The electrodes 12 are arranged in a pattern which allows a number of relatively long conduction paths through the interior of the kidney K, so as to increase the probability that a region R (described above) will have an effect on a given electrode-to-electrode measurement when used in unipolar mode.

For example, nominal path “P1” extends from electrode 12A to a second electrode 12B, corresponding to a path originating near a north or upper pole “N” of the kidney K and terminating near a south or lower pole “S” thereof. Nominal path “P2” extends from the first electrode 12A to a third electrode 12C. Nominal path “P3” extends from the first electrode 12A to a fourth electrode 12D. Finally, nominal path “P4” extends from the first electrode 12A to a fifth electrode 12E. The illustrated paths P1-P4 presume the use of the first electrode 12A as an introduction point for an electrical signal; however any of the electrodes 12 may be used for this purpose. For example, a path P5 extends from the second electrode 12B to the third electrode 12C. This provides a multiplicity of paths even with a relatively small number of electrodes 12. Of course, the internal structure of the kidney K is not homogenous, and thus the actual conduction path between any two electrodes 12 will vary and may not be linear.

The number, type, and position of the electrodes 12 may be varied to suit a particular application. In this example, the paths P1-P4 pass through and correspond to arbitrarily-designated quadrants of the kidney K labeled I-IV. This provides adequate signal coverage for the entire kidney K. This also allows the isolation of specific areas of the kidney K by the use of individual electrodes or pairs of electrodes 12. For example, If a particular signal pattern is observed only at a single electrode 12B (in a multipolar mode, along path P6) but not at other electrodes 12, this would indicate a difference in the structure of the Kidney K at that location. If a signal pattern is observed only between electrodes 12B and 12C (in a unipolar mode, along path P5) but not between other electrode pairs, this would indicate a difference in the structure of the kidney K somewhere in quadrants I or II. The greater the number of electrodes 12 used, the more accurate the location data will be. This aspect of the network of electrodes 12 may also be used to track the progression of changes in condition over a series of spaced-out observations.

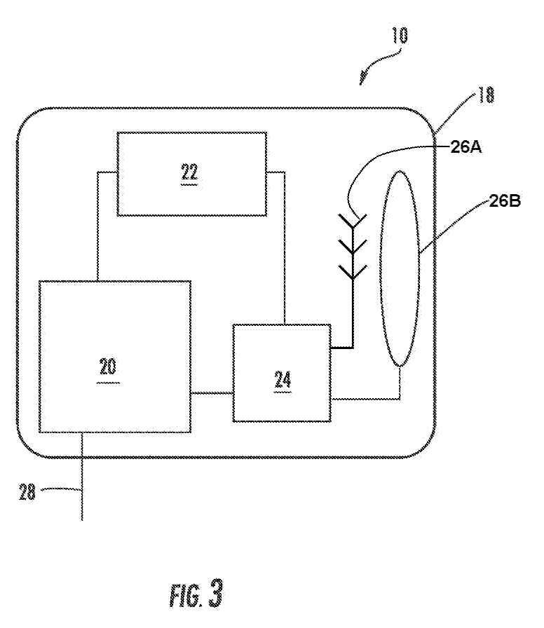

The sensor unit 10, shown in more detail in FIG. 3, is structured to be implanted into the patient's body and includes a housing 18 constructed of a material that is biologically inert, such as titanium or silicone. The sensor unit 10 includes a controller 20 such as a microprocessor operating under software control or a programmable logic controller (PLC), an energy source 22 (e.g. a storage battery), a transceiver 24, and a transducer (generally referred to at 26). The energy source 22 provides electrical or thermal energy to the other components of the sensor unit 10. The transceiver 24 and transducer 26 are configured to communicate with a compatible transducer (not shown) an external device such as a relay or data unit (described below). Such communication may be through radio frequency (RF), in which case the transducer would be a conventional antenna, shown at 26A, or through inductive coupling, in which case the transducer would be an induction coil, shown at 26B. If an inductive coupling is provided, either as part of the transducer 26 or separately, it may also be used to provide power to the sensor unit 10 or to recharge the energy source 22, or both. Multiple sensor units 10 may be provided for multiple organs.

The controller 20 is connected to terminals 28 which are in turn connected to the leads 14. The controller 20 is capable of generating a voltage having a desired characteristic, such as AC, DC, or an arbitrary waveform, which is injected through a selected one of the electrodes 12. The controller 20 is able to receive or register the returned signal from one or more of the electrodes 12, and to output corresponding analog or digital data which allows calculation of the impedance between electrodes. The controller 20 may also include hardware, software, or a combination of the two operable to calculate impedance values directly. These functions may be done either on demand in response to an external command, or automatically at a programmed time interval. The impedance measurement or other signal aspect may then be transmitted to an external device through the antenna 26. The operation of the sensor unit 10 is explained more fully below.

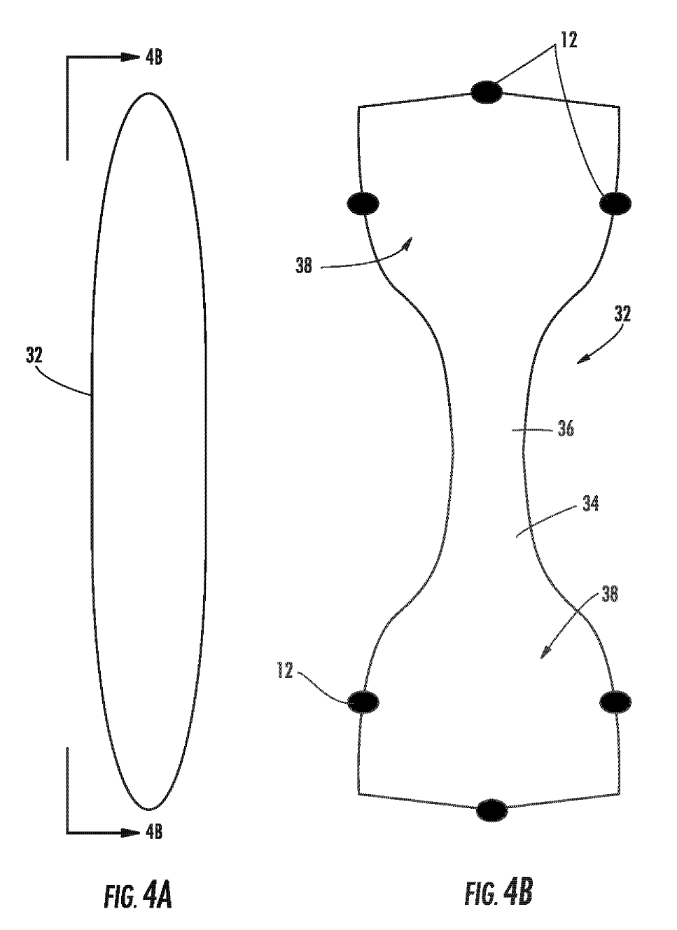

The electrodes 12 may be physically connected to the kidney K in a number of ways. For example, a network of known screw-in or suture-in electrodes, such as the type used for conventional pacemaker lead connections, may be individually attached to the Kidney K. Alternatively, a mesh, net, sock, or other structure which holds several electrodes 12 in a desired configuration may be provided. For example, FIGS. 4A and 4B illustrate a sensor sock 32. The sock 32 comprises a body 34 which carries several electrodes 12 arranged in a pattern to provide the selected conduction paths as described above. While only a few electrodes 12 are illustrated, The sensor sock 32 may include a large number of closely-spaced electrodes 12 so as to facilitate localized sensing. The body 34 may be constructed as a band, envelope, or other flexible structure. In this example, the body 34 is a continuous loop with a pinched waist 36 and enlarged ends 38. The body 34 may be constructed from any flexible biocompatible material, such as biocompatible polymers, or natural or synthetic woven or nonwoven fabrics. The body 34 is generally an electrical insulator so as to avoid unwanted conduction between the electrodes 12. Some degree of elasticity is useful in helping the body 34 conform to the kidney K.

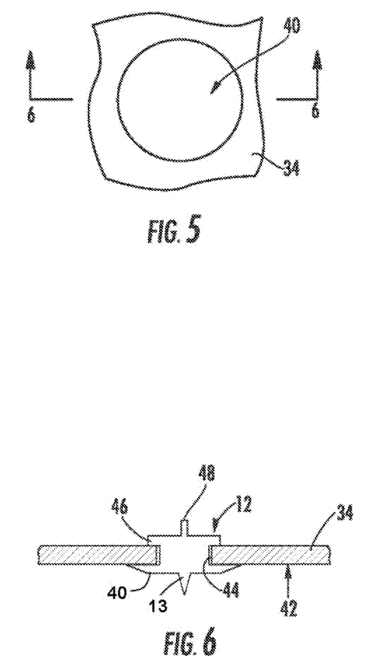

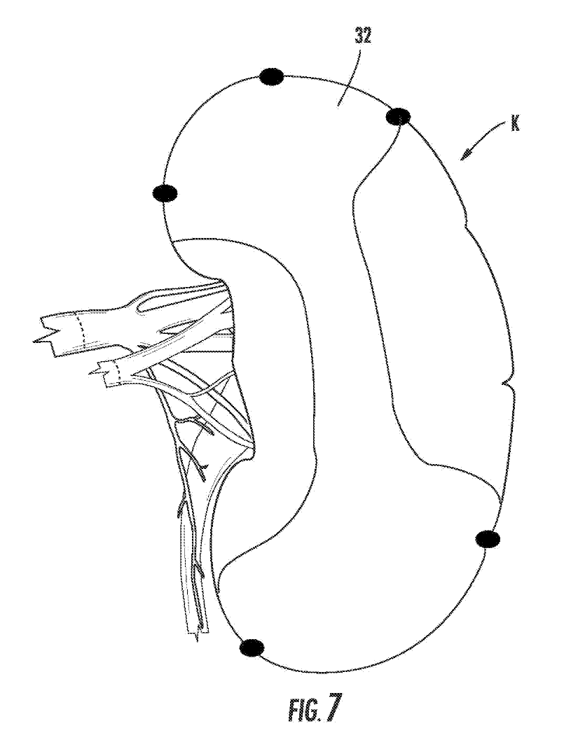

FIGS. 5 and 6 illustrate one of the electrodes 12, showing how it may be attached to the body 34. This arrangement is typical of all of the electrodes 12. The electrode 12 has a conductive face 40 which lies against the inner surface 42 of the body 34, and is held in place in an opening 44 by a crimped or formed flange 46. A terminal 48 extends outwardly from the electrode 12. The electrode 12 may optionally include a pointed tip 13 or other invasive structure extending from the conductive face 40.

The sensor sock 32 is shown applied over a kidney K in FIG. 7. It may be held in place with stitches or sutures, staples, adhesives, or simply by elastic tension.

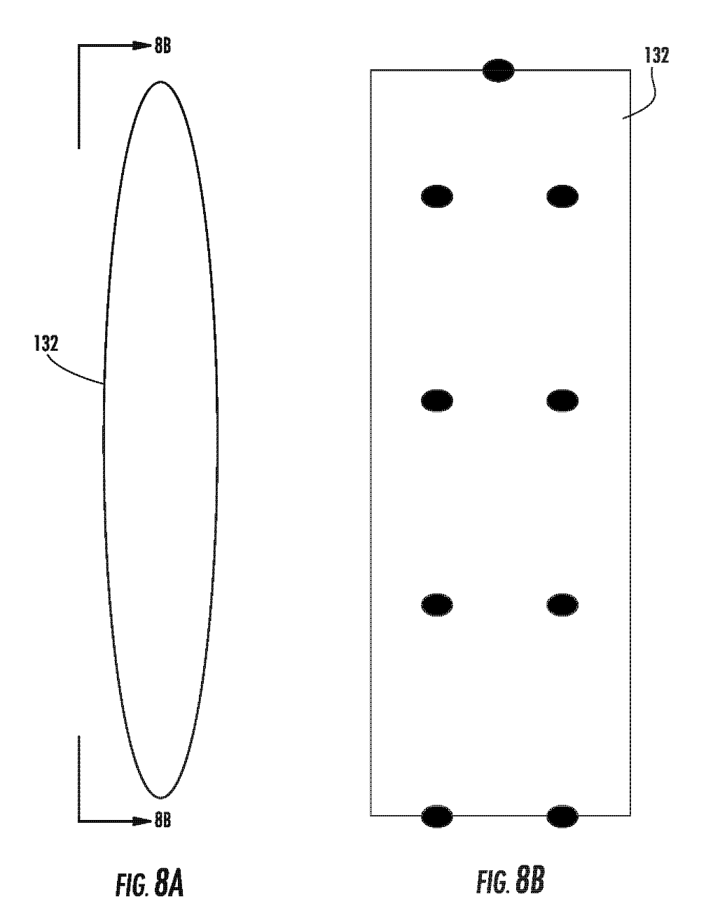

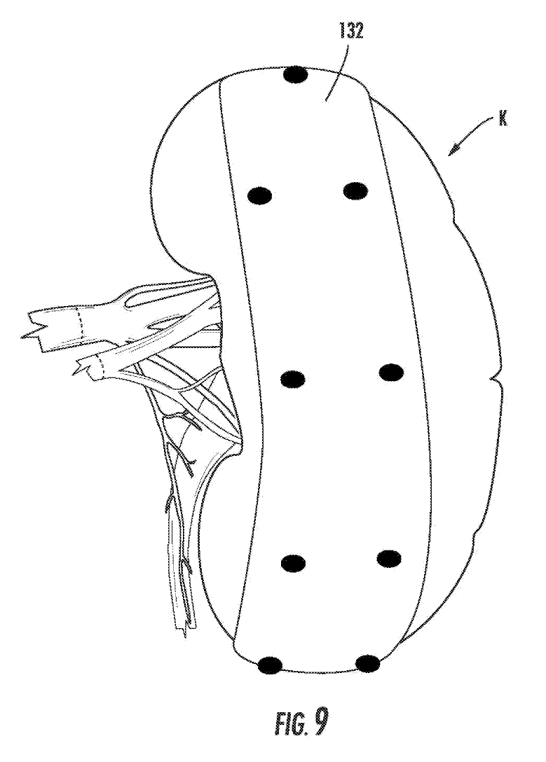

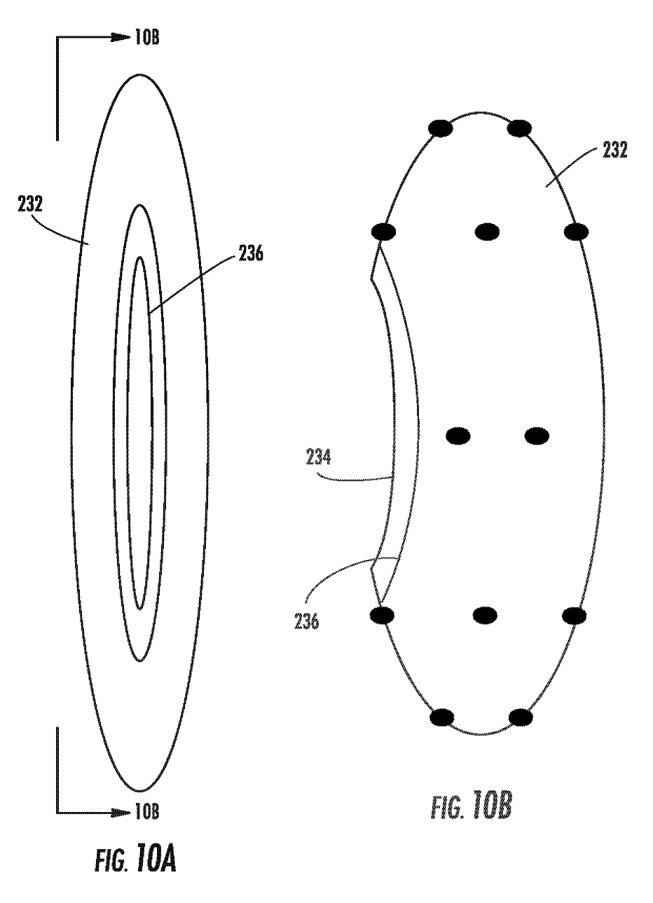

FIGS. 8A and 8B illustrate an alternative sensor sock 132 which is similar in construction to the sensor sock 32 but is rectangular in side view. It is shown applied over a transplanted kidney K in FIG. 9.

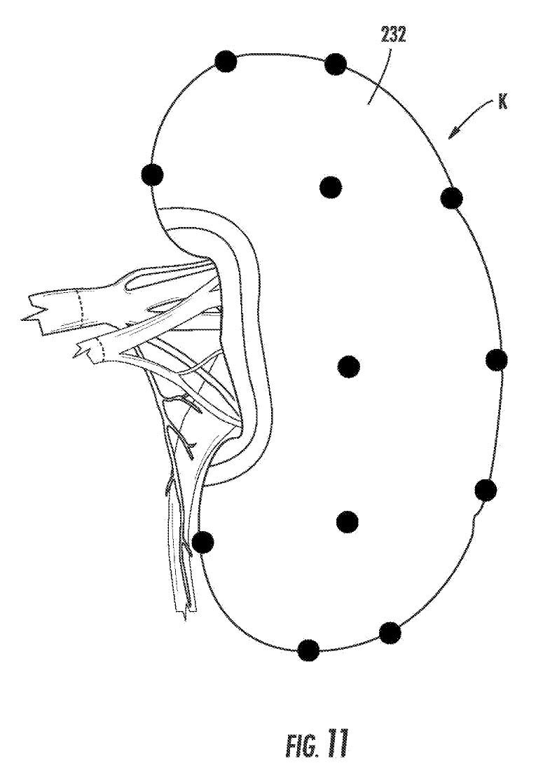

FIGS. 10A and 10B illustrate yet another alternative sensor sock 232 which is similar in construction to the sensor sock 32. The sensor sock 232 is shaped like a closed envelope or pouch with an opening 234 optionally surrounded by elastic banding 236. This shape allows the sensor sock 232 to be stretched over a kidney K and held in place entirely by elastic tension, as shown in FIG. 11.

In addition to, or as an alternative to the impedance measurements described above, the sensor unit 10 may be used to produce a selected electrical waveform, which is introduced through one or more of the electrodes 12. The selected waveform may be sinusoidal, square-wave, sawtooth, half-wave DC, or it may be an arbitrary waveform compounded from individual signal elements. The selected waveform may be introduced a single time during a session or as a repeating sequence. The selected waveform is modified in various ways depending upon its path through the kidney K, and is subsequently picked up by at least one of the electrodes 12. For example, if the electrode 12 is being used in a multipolar mode, the signal will be sensed by that same electrode 12. If the electrode 12 is being used in a unipolar mode, the signal will be sensed by another electrode 12. The modified waveform (in the form of an electrical signal) is then output by the sensor unit 10 to an external device for subsequent analysis.

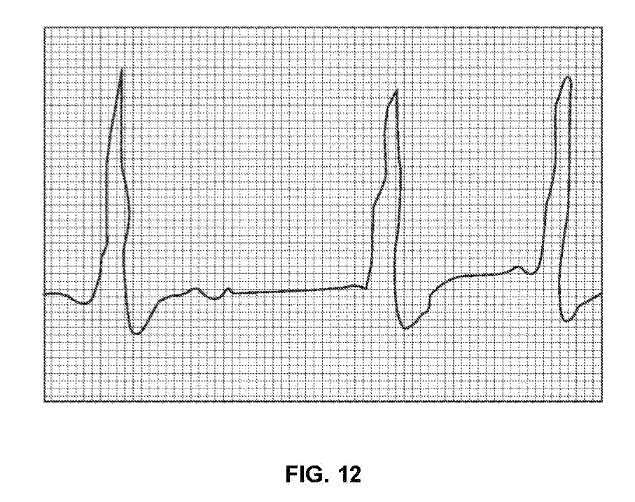

Analysis software, for example running on either a local data unit or a data server (described below), is used to analyze the data received from the patient's kidney K in several ways. According to one procedure, each time data is received corresponding to the electrical signals received by the sensor unit 10, the analysis software digitally creates or generates a graphical representation of the modified waveform such as a graph, or chart (referred to herein as a “waveform”). An example of a waveform is illustrated in FIG. 12. In accordance with conventional electrogram practice, the horizontal axis of this waveform represents a time scale (e.g. seconds), and the vertical axis of the waveform represents amplitude (e.g. Volts or millivolts). For example, the electrical signals received from the sensor unit 10 may be analog signals that are digitized (e.g. at 1 KHz with a 8-bit resolution). Regardless of what type of signal is used, a baseline or reference electrical signal may be obtained using the above-referenced procedure to produce a reference waveform that is then stored for later analysis. The reference waveform can be obtained when the patient undergoes the kidney transplant, when the sensor unit 10 is implanted, or at some other predetermined time.

Several techniques may be used to generate the waveforms or portions thereof so as to produce data which is “cleaner” than the raw digitized data, i.e. relatively free from effects of electrical noise or digitization errors, and easier to analyze.

For example, depending on the waveform selected, it may comprise a series of line segments or portions having a high slope or first derivative, such that peaks (and nadirs) occur as sharply delineated events (i.e. the curves are strongly convex). Accordingly, peak detection may be implemented by establishing a minimum slope value. To accomplish this, the entire waveform is evaluated, either by the analysis software or by separate pre-processing software, for the presence of any location where the slope is less than the minimum value. Each of these locations are identified as a peak. Suitable threshold values will depend upon the particular waveform that is selected.

Some selected waveforms may appear as deviations from a baseline or generally horizontal trace, which may or may not be equal to a zero electrical potential line. The value (i.e. voltage level) of the baseline affects other measurements such as baseline-to-peak amplitude and area under the curve (described in more detail below). The specific value of the baseline is calculated based on the specific equipment configuration.

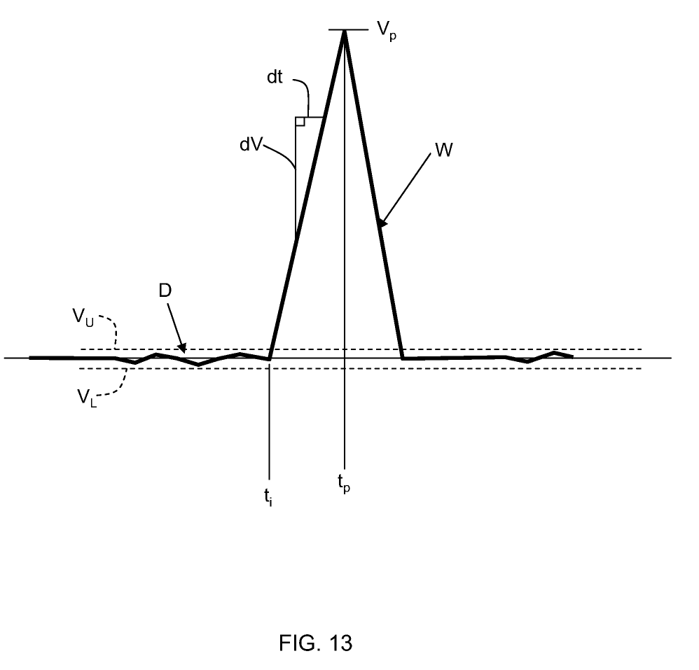

In practical application, portions of the signal ahead and behind of the selected waveform shape will not match the established baseline, i.e. they will not be simple horizontal traces, but will rather exhibit many small deviations. This is depicted by arrow “D” of the exemplary waveform “W” in FIG. 13. In order to reduce the uncertainty in several measurements caused by this variation, a deadband or hysteresis band with preselected upper and lower voltage limits “V_{U}” and “V_{L}” may be applied to the waveform W. For purposes of analysis, the beginning (or end) of the selected waveform is assumed to begin or end at the time value tᵢ which the upslope or downslope of the waveform W intercepts the relevant limit V_{U} or V_{L}.

One manner in which the hysteresis band intercept may be accurately located is to apply a linear slope calculation to the relevant portion of the waveform W. For example, using the peak detection method described above, the time tₚ at which the dominant peak occurs and the peak voltage Vₚ will be known. The slope dv/dt of the immediately preceding segment is then determined, by calculating a linear ratio using an appropriate dt (e.g. 1 ms if a 1-kHz sampling rate is being used). Once the slope is known, it may be extrapolated back to calculate the intercept time tᵢ, for example using equation (1) below. The resulting time tᵢ is taken to the be the “beginning” of the upslope. A similar procedure may be used to determine the intercept of other upslopes or downslopes within the waveform W.

tᵢ=tₚ−(vₚ−vᵤ)(dv/dt)⁻¹ (1)

In cases where a cyclic signal or repeating waveform is used, the reference waveform and the registered waveforms which are generated for comparison and analysis purposes may be representative of the average of many individual waveforms in the data population recorded during a period of data collection.

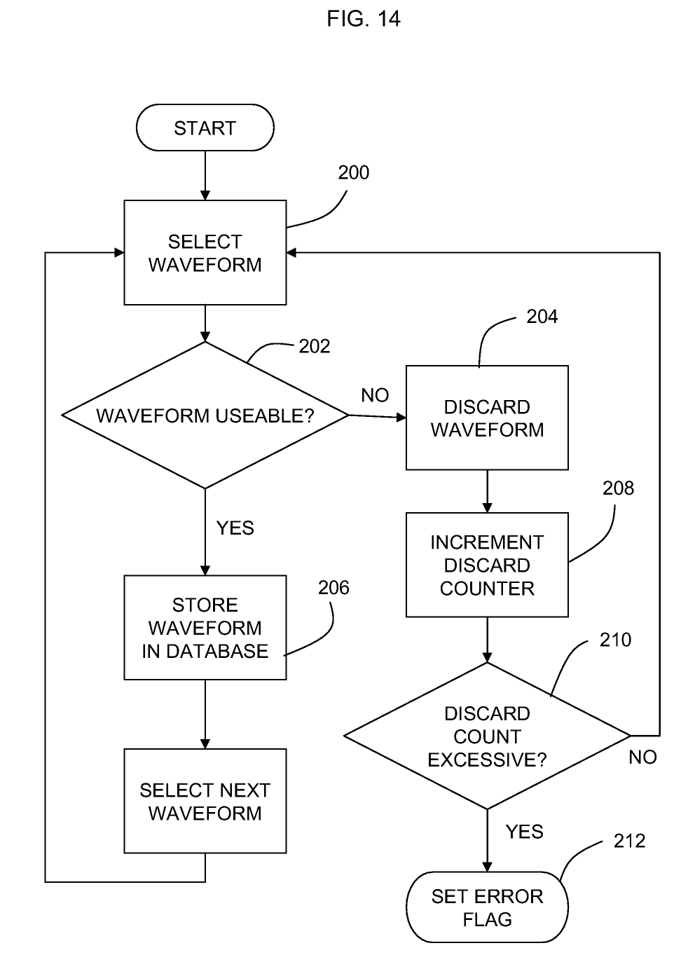

The preprocessed data is evaluated as follows, with reference to FIG. 14. First, a waveform resulting from a data collection session is selected (block 200) to be observed and evaluated against a predetermined standard in block 202. This process can occur in real-time as the waveforms are collected, or it may be applied to a set of waveforms which have been temporarily stored. If the waveform does not meet the applicable standard it is deemed “not usable”. It is discarded (block 204) and not used in the generation of the averaged waveform, as described below. The purpose of this initial step is to serve as a gross check on the quality of the data and to prevent outlying data from corrupting the data population, possible leading to incorrect diagnosis. If the waveform is usable, it is stored in a statistical database (block 206), or marked for permanent storage.

Various techniques may be used to implement this step. For example, waveforms elements which are at the edges of a normal distribution (e.g. beyond a 2σ interval) in terms of one or more characteristics such as area, amplitude, or slew may be discarded and not used in the generation of the averaged waveform.

Another method for eliminating extraneous data is to test a defined point-to-point horizontal distance (e.g. peak-to-peak or baseline-to-baseline) of each waveform. If any one waveform has a point-to-point distance varying from the average point-to-point distance by more than a selected threshold value, for example plus or minus 5%, then that entire waveform would be discarded and not used in the generation of the averaged waveform.

As the data is initially tested, a counter is incremented (block 208) each time a waveform is discarded. A high value of this counter could indicate an equipment fault or human error in collecting the data. High values may also indicate extreme acute rejection. Accordingly, this counter serves as a gross check for allograft rejection. If the counter exceeds a predetermined standard at block 210, the process is stopped and an error flag is set for operator attention at block 212. The process is repeated until all of the waveforms in the data collection session have been evaluated.

Next, the remaining waveforms from the data collection session are used to construct a single average waveform. The initial waveform generated at the reference time, immediately or very shortly after transplantation, becomes the reference waveform described above. Each subsequent data collection session results in a new averaged registered waveform. For example, a data collection session may be conducted three times each day after transplantation, resulting in three new registered waveforms each day.

When creating a representative waveform, the “average” image may be created in two different ways. In a first exemplary technique, all of the non-discarded waveforms recorded are averaged together to generate a single average waveform.

In another exemplary technique, the individual elements described above are identified for each non-discarded waveform in the data population. Those individual elements are averaged together. Then the individually-averaged elements are assembled to form a composite waveform.

Various portions, features, or elements of the waveforms can be used as a basis for comparison between the reference waveform and the registered waveform in determining the presence or absence of rejection.

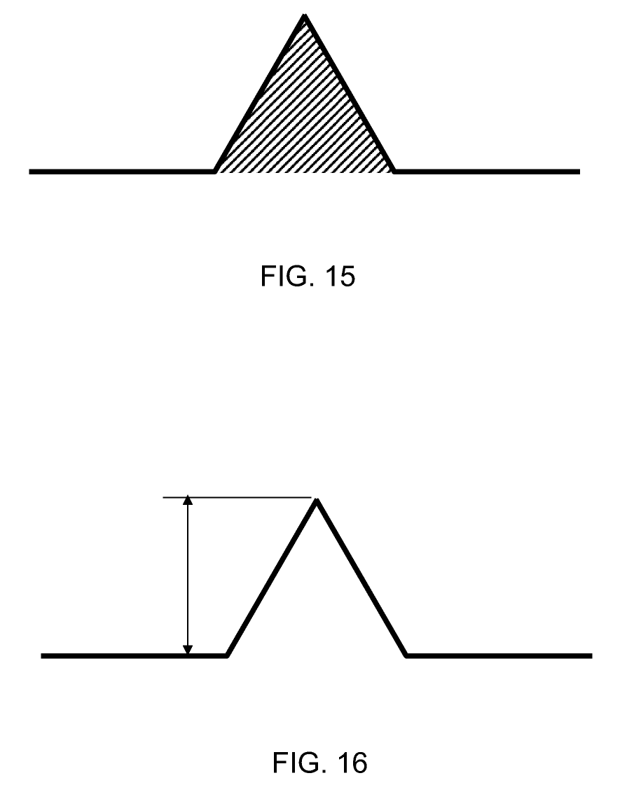

One element is area measurement, which is shown in FIG. 15 in which the area being measured in shaded for identification. Known techniques of numerical integration are used to implement the area measurement.

Another element is amplitude measurement. For example, FIG. 16 illustrates baseline to peak amplitude measurement. These values are measured in millivolts (mV).

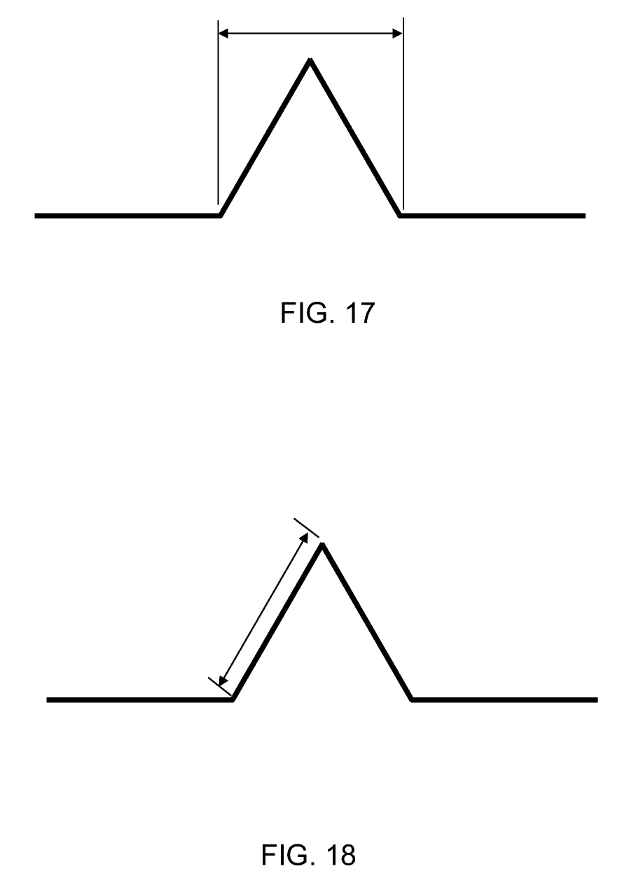

Another element is duration. FIG. 17 illustrates the measurement of total waveform duration (i.e. baseline-to-baseline). The total waveform duration is measured in milliseconds (ms).

Another element is slew rate or slope. FIG. 18 illustrates the measurement of the a selected peak upslope. The slew rate is measured in volts per second (V/s).

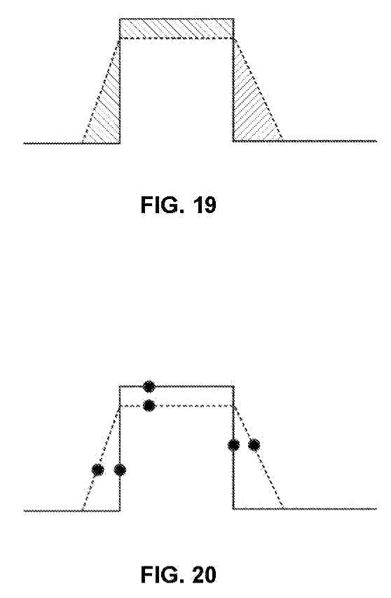

The difference in one or more of the individual signal elements or measurements described above (e.g. area, amplitude, slew, or impedance) between the reference waveform and the registered waveform is measured and used to assess organ function. The waveforms may also compared by measuring the total area of discrepancy between the waveforms and determining a comparison percentage match, as shown in FIG. 19, or by a point-to-point comparison, as shown in FIG. 20.

Regardless of which elements are being compared, a match-percentage threshold may be established, based on correlation to clinical data, which would indicate acute heart rejection. If a waveform corresponding to an electrical signal received from the patient's organ does not correlate to the reference waveform, by an amount greater than or equal to the established match-percentage threshold, then rejection is present. Early detection of rejection advantageously permits prompt initiation of life saving therapy.

Alternatively, evaluation of the waveforms may be carried out based on a multivariable statistical analysis of shifts in the registered data. When the average registered waveforms are created, each new waveform, along with the values of all of its individual elements, becomes a member of a statistical population in a database. As rejection takes place, causing changes in the kidney K, it is expected that the individual waveform elements described above will change in different ways. For example, the R-wave upslope might increase while the peak-to-peak amplitude decreases. No one of these elements necessarily represents a simple rejection-specific parameter, rather the aggregate difference, or certain combinations of changes, represents allograft rejection. However, the aggregate effect of these changes can be correlated to the presence of rejection.

Under either of the methods described above, a scale of rejection can be created. The greater the deviation from a nominal condition (determined either statistically or in terms of a scalar measurement), the more likely actual rejection is taking place, or the greater the severity of rejection. Increasing numbers on a scale is indicative of greater deviation of the registered waveform data from the reference waveform. The numbers on the scale may be likened to “grades” of rejection.

It is also possible correlate the scale of rejection to clinical results (from biopsies, autopsies, etc.) and to established “grades” of allograft rejection.

The method described herein is able to detect very slight changes in the recorded data. As such, it is believed that changes in the kidney K measurable on the scale of rejection will be present even if no rejection is yet observable in a contemporaneous biopsy. This can occur because the method described herein is sensitive to changes throughout the structure of the kidney, while a biopsy may show negative results if it is not taken from a localized area that happens to site where rejection is just starting. The present method thus has the possibility of detecting rejection early enough so as to be “predictive” in nature when compared to biopsies. Early detection of rejection advantageously permits prompt initiation of life saving therapy. This early detection is especially important in immuno-compromised patients who are prone to rapid onset of acute rejection.

In another aspect of the invention, which may be used separately or in conjunction with the electrical signal monitoring method described above, rejection and function of a transplanted kidney or other organ may be determined by monitoring flow rates of blood into and out of the organ, or other flow characteristics.

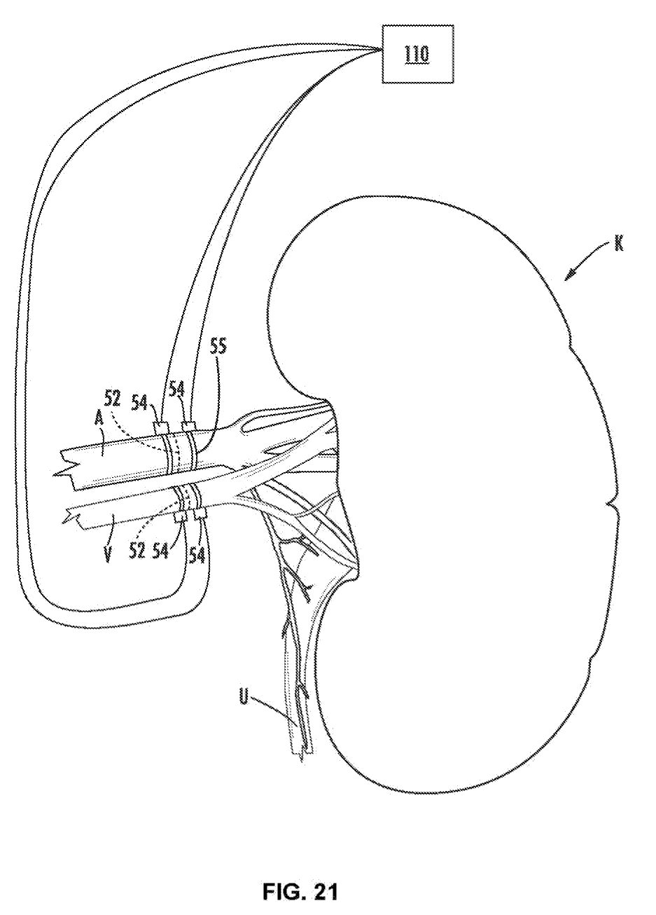

FIG. 21 illustrates a transplanted kidney “K” showing some of its structures including a stub portion of a renal vein “V”, a stub portion of a renal artery “A”, and ureter “U”. During a transplant procedure, these stub portions of the renal vein V and renal artery A are joined with the renal vessels of the host body by anastomoses 50 and 52 (indicated generally with dashed lines). Anastomosis is the surgical connection of tubular structures to form a continuous channel and may be done by suturing, stapling, adhesives, or the like. This process is not perfect and can lead to leakage of blood into the peritoneal cavity, with subsequent failure of the kidney K.

The quality of the anastomoses 50 and 52, the status of the kidney K, or both may be determined by monitoring flow through the renal vessels. FIG. 21 illustrates a kidney K having one or more flow transducers 54 disposed in contact with or in close proximity to the renal vessels, for example using straps 55, and coupled to an implanted sensor unit 110 through leads 112.

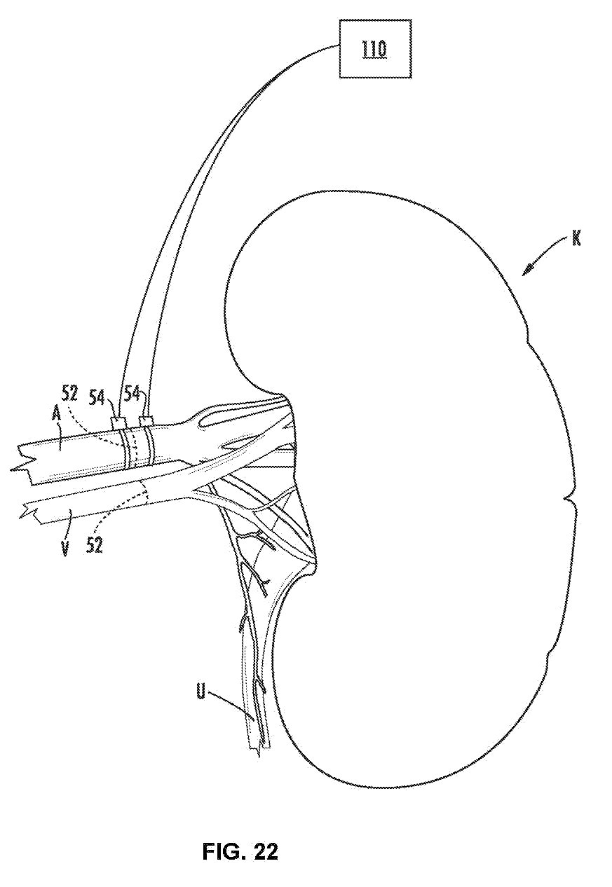

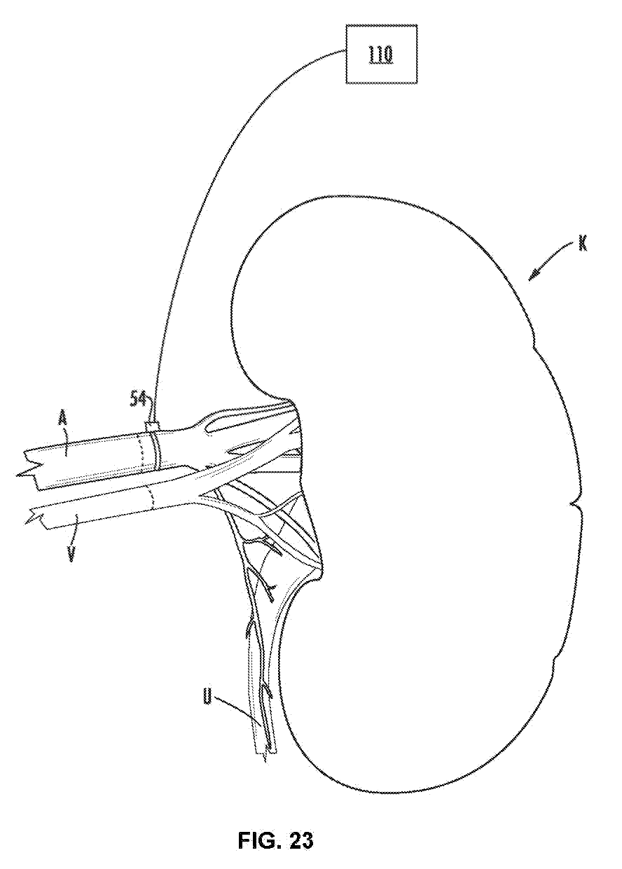

The flow transducers 54 may be active or passive. In this example, they are active ultrasonic flow sensors of a known type which are capable of imparting a sound wave into a vessel and sensing the sound energy reflected by bubbles or particles suspended in the blood. Such sensors are capable of measuring the diameter of the vessel to which they are attached. This information, in conjunction with an observed or assumed average blood velocity, may be used to calculate a flow rate within the vessel. In FIG. 21, a first pair of flow transducers 54 are attached to the renal artery A on opposite sides of the anastomosis 52, and a second pair of flow transducers 54 are attached to the renal vein V on opposite sides of the anastomosis 50. Other types of transducers which sense flow characteristics that can be related to flow (e.g. pressure, vessel diameter) may also be used. The transducers 54 may be attached with bands 56 as shown, or with stitches, sutures, or adhesives. FIG. 22 shows a different configuration in which a single pair of flow transducers 54 are attached to the renal artery A on opposite sides of the anastomosis 52. The single pair could be applied to the renal vein V instead. FIG. 23 shows yet another variation in which a single flow transducer 54 is attached to one of the renal vessels (in this case, the renal artery A).

The sensor unit 110 may be substantially identical in construction to the sensor unit 10 described above, for example it may include a housing which contains a controller, energy source, transceiver, and antenna (not shown). The sensor unit 110 is capable of sending a signal to the flow transducer 54, which then inputs sound energy into the renal vessel. The sensor unit 110 is able to receive and optionally store the reflected signal returned from the flow transducer 54, either on demand in response to an external command, or automatically at a programmed time interval. The reflected signal may then be transmitted to an external receiver. The operation of the sensor unit 110 is explained more fully below. In a combined application, a single sensor unit 110 may be used to collect information both from electrodes 12 and flow transducers 54.

The flow transducers 54 may be employed in various ways to produce clinically useful information. For example, if a flow transducer 54 is placed on each side of the anastomosis 52, as shown in FIG. 22, a flow rate through the renal vessel (in this case the renal artery A) may be measured using known Doppler frequency shift measurement techniques. If there is no leakage, the flow rate on both sides of the anastomosis 52, whether the flow is continuous or pulsatile, should be identical. If there is any significant difference in the flow rates, this would indicate the presence of a leakage flow from the renal artery A into the peritoneal cavity. Using this same technique with the four-transducer configuration shown in FIG. 21, the leakage may be independently monitored in the renal artery A and the renal vein V.

Alternatively, using at least one flow transducer 54, as shown in FIG. 23, a flow comparison may be used to evaluate the status of the kidney K. In this method, the flow transducer 54 is used to determine a reference flow rate. The reference flow rate can be obtained when the patient undergoes the kidney transplant, when the sensor unit 110 is implanted, or at some other predetermined time. Additional flow measurements are then taken at subsequent times. A significant change in the flow rate would be indicative of kidney rejection.

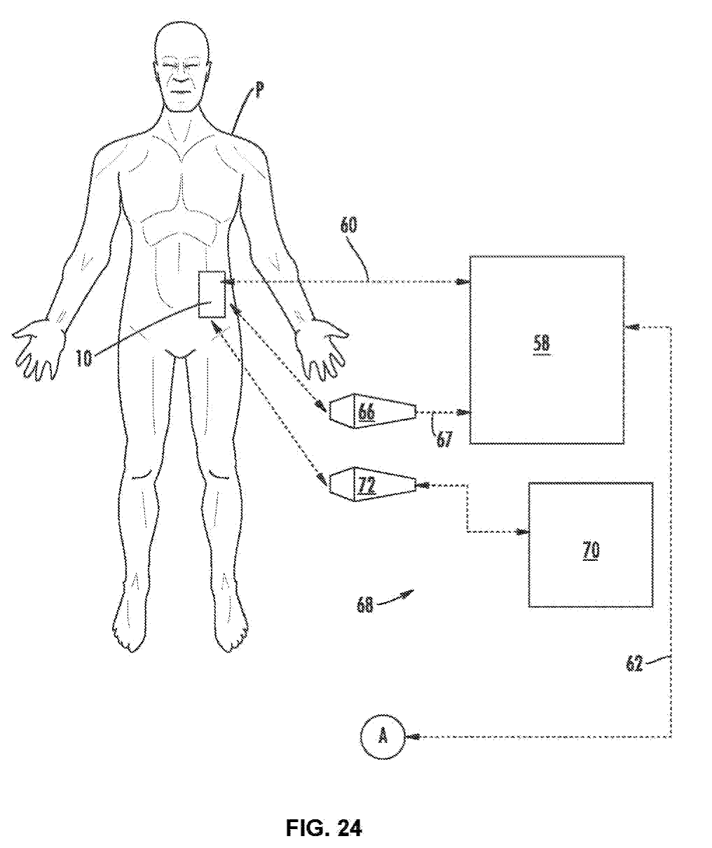

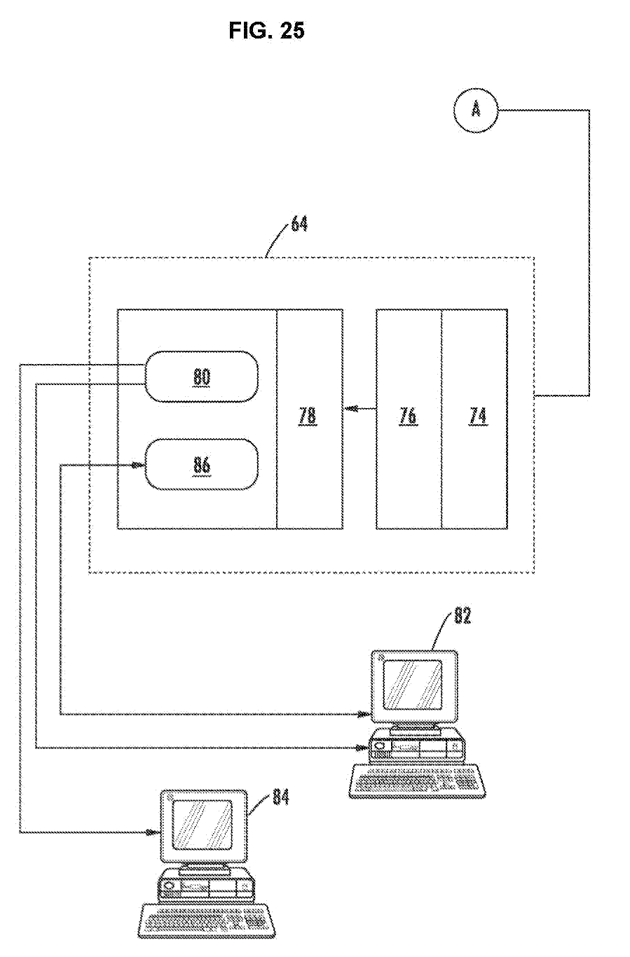

FIGS. 24 and 25 illustrate a data collection system which may be used with either of the monitoring methods described above. The system includes a sensor unit (e.g. sensor units 10 or 110 described above), implanted into a patient P.

A local data unit 58 is used to receive, store, and optionally process data from the sensor unit 10. The local data unit 58 can include a computer, microprocessor, or central processing unit operating under software control, with associated data storage means such as flash memory, RAM, EEPROM, hard disk, floppy disks, CD or DVD-ROM, etc., and a transceiver or other data communication means (e.g. a TCP/IP network adapter or modem).

In use, the local data unit 58 is placed in communication with the sensor unit 10, for example using a relay unit 66 such as the illustrated handheld wand. The relay unit 66 includes an antenna, power source, data storage means, and transceiver compatible with the with that of the sensor unit 10 (such as an induction coil). In use, the relay unit 66 receives data from the sensor unit 10, for example by inductive coupling at short range. The data is then either stored for later transfer to the local data unit 58, or immediately transferred to the local data unit 58. The transfer occurs through a communications link 67 such as a cable, infrared transmitter, or wireless link (e.g. BLUETOOTH wireless protocol).

Optionally, communication between the sensor unit 10 and the data unit 58 may be through a radio frequency (RF) communications link 60 of a known type.

The local data unit 58 receives data from the sensor unit 10 and then transfers that data over a remote communications path 62 such as a wireless or wired packet-switched network (e.g. a local area network, a wide area network, or Internet), over telephone lines using a modem, or through satellite connection. The remote communications link may be encrypted for security purposes. The data is then received by a data server 64 at a remote location (see FIG. 25). Optionally, the data may be received from the sensor unit 10 and then stored by the local data unit 58 for later transmission to the data server 64.

Optionally, a physician interface unit 68 may be provided. This comprises a computer 70 (e.g. a laptop microcomputer) and a relay unit 72 similar to the relay unit 66 described above, or other suitable communications link compatible with the sensor unit 10. The physician interface unit 68 is programmed with software enabling it to receive data from the sensor unit 10 and display the data for review, for example to show graphically in real time the impedances, flow rates, or other data measured and transmitted by the sensor unit 10. It may also be programmed to calculate impedance values based on the received data and/or perform the data analysis described above. The physician interface unit 68 is also able to send instructions to the sensor unit 10 through the relay unit 72, for example to change the value of programmable parameters of the sensor unit 10 (such as a measurement interval), to interrogate the sensor unit 10 for the actual values of the programmable parameters, or to command the sensor unit 10 to transmit data.

FIG. 25 illustrates the data server 64 and related components. The data server 64 receives data from the local data unit through the communications path 62 described above. A data receiving software module 74 may be provided for this purpose. The data may then processed by an analysis software module 76 which is capable of performing the impedance calculations, reference-waveform comparisons, and/or signal analysis described above. The processed data is stored in a database 78, such as a structured query language (SQL) database. The data may then be accessed by an electronic medical records (EMR) software module 80 which permits a user to view summaries of patient data, graphical analysis screens, and the like. The EMR module 80 may be accessed by a monitoring service at a remote computer 82 (e.g. over a secure network connection), or by another authorized user, such as a patient's primary care physician, again at a remote computer 84 which communicates with the data server 64 by a network connection. A billing software module 86 may also be provided within the data server 64 to track usage by the monitoring service or other authorized user.

The foregoing has described systems and methods for monitoring organs. While specific embodiments of the present invention have been described, it will be apparent to those skilled in the art that various modifications thereto can be made without departing from the spirit and scope of the invention. Accordingly, the foregoing description of the preferred embodiment of the invention and the best mode for practicing the invention are provided for the purpose of illustration only and not for the purpose of limitation, the invention being defined by the claims.

What is claimed is:

1. A method for monitoring a patient's organ, comprising:
(a) inputting an electrical signal into the organ at a first location;
(b) receiving the electrical signal from the organ at a second location spaced-apart from the first location; and
(c) comparing the received electrical signal to a reference electrical signal to determine whether the patient's organ is functioning properly.

2. The method of claim 1 wherein the organ is a kidney.

3. The method of claim 1 wherein the signal is representative of an impedance of the organ.

4. The method of claim 1 wherein the comparing step comprises:
(a) generating a first waveform corresponding to the reference electrical signal;
(b) generating a second waveform corresponding to the received electrical signal; and
(c) measuring an area between the first waveform and the second waveform to determine whether the patient's heart is functioning properly.

5. The method of claim 1 wherein the comparing step comprises:
(a) generating a first waveform corresponding to the reference electrical signal;
(b) generating a second waveform corresponding to the received electrical signal;
(c) identifying a plurality of comparison points for the first waveform;
(d) identifying a plurality of comparison points for the second waveform, each of the plurality of comparison points for the second waveform corresponding to one of the comparison points for the first waveform; and
(e) measuring differences between each of the corresponding plurality of comparison points for the first waveform and the second waveform to determine whether the patient's heart is functioning properly.

6. A method for monitoring a patient's organ, comprising:
(a) measuring a first flow characteristic within a blood vessel which is connected to the organ; and
(b) comparing the first flow characteristic to a reference flow characteristic to determine whether the patient's organ is functioning properly.

7. The method of claim 6 wherein the organ is a kidney.

8. The method of claim 6 wherein the first flow characteristic is a fluid flow rate within the vessel.

9. The method of claim 6 wherein the blood vessel is joined by an anastomosis on a first side of the anastomosis, and the first flow characteristic is measured on a first side of the anastomosis, and wherein the reference flow characteristic is a flow characteristic measured in the blood vessel on a second side of the anastomosis.

10. The method of claim 6 wherein the step of measuring a first flow characteristic is carried out by:
(a) inputting a first sound wave into the blood vessel;
(b) receiving a first reflected sound wave from the blood vessel;
(c) generating a first electrical signal in response to the reflected sound wave.

11. A system for monitoring a patient's organ, comprising:
(a) A sensor sock comprising a flexible body adapted to at least partially surround an organ, the sock carrying a plurality of spaced-apart electrodes; and
(b) a sensor unit adapted to be implanted into the patient's body, the sensor unit connected to the electrodes and adapted to transmit and receive electrical signals from the electrodes.

12. The system of claim 11 wherein at least two of the electrodes are disposed at opposed ends of the sock.

13. The system of claim 11 wherein the sock is at least partially elastic.

14. The system of claim 11 wherein the sensor unit comprises:
(a) a controller operable to transmit electrical signals to and receive electrical signals from the electrodes;
(b) a transceiver operably connected to the controller; and
(c) an transducer operably connected to the transceiver.

15. The system of claim 11 wherein one of the electrodes is a signal electrode adapted to transmit a signal; and
a plurality of the electrodes are sensor electrodes adapted to receive a signal originating at the signal electrode;
The sensor electrodes being disposed in a spaced-apart arrangement to as to permit propagation of electrical signals throughout substantially all of interior of the organ.

16. The system of claim 15 further comprising a computer programmed to receive the electrical signals from the sensor unit corresponding to a received electrical signal and to compare the registered electrical signal to a reference electrical signal to determine whether the patient's organ is functioning properly.

17. The system of claim 16 further comprising a local data unit configured to receive data from the sensor unit and to transmit the received data to the computer.

18. The system of claim 17 further comprising a relay unit adapted to receive data from the sensor unit through inductive coupling therewith and to transmit the received data to the local data unit.

19. A system for monitoring a patient's organ, comprising:
(a) At least one transducer adapted to be attached to a blood vessel connected to the organ, and to sense at least one characteristic of flow inside the blood vessel; and
(b) a sensor unit adapted to be implanted into the patient's body, the sensor unit connected to the transducer and adapted to transmit and receive electrical signals from the transducer.

20. The system of claim 19 wherein the transducer comprises an ultrasonic sensor operable to:
(a) inputting a sound wave into the blood vessel;
(b) receiving a reflected sound wave from the blood vessel; and
(c) generating an electrical signal in response to the reflected sound wave.

21. The system of claim 19 further comprising a computer programmed to receive the electrical signals from the sensor unit corresponding to a received electrical signal and to compare the registered electrical signal to a reference electrical signal to determine whether the patient's organ is functioning properly.

22. The system of claim 19 further comprising a relay unit adapted to receive data from the sensor unit through inductive coupling therewith and to transmit the received data to the local data unit.

23. A system for monitoring a patient's organ, comprising:
(a) a sensor unit adapted to be implanted into the patient's body, and to register an electrical signal from the patient's organ; and
(b) a local data unit in operable communication with the sensor unit, the local data unit configured to receive and store data from the sensor unit and to selectively transmit the received data over a communications path.

24. The system of claim 23 further comprising a computer programmed to receive the electrical signals over the communications path corresponding to a registered electrical signal, and to compare the received electrical signal to a reference electrical signal to determine whether the patient's organ is functioning properly.

25. The system of claim 24 wherein the computer is a server including:
(a) a data receiving software module configured to receive data from the local data unit;
(a) an analysis software module configured to compare the registered electrical signal to a reference electrical signal to determine whether the patient's organ is functioning properly;
(b) a database configured to store data processed by the analysis software module; and
(c) an electronic medical records software module configured to provide access to the data stored in the database.

26. A method of monitoring a transplanted organ, comprising:
(a) during a first data collection session occurring at a reference time, injecting a predetermined electrical signal into a patient's organ;
(b) during the first data collection session, registering a resulting electrical signal from the organ, the resulting electrical signal configured as a first series of waveforms;
(c) generating from the first series of waveforms, a reference waveform representative of the average characteristics of the waveforms collected during the first data collection session;
(d) during a subsequent data collection session occurring at a time subsequent to the reference time, injecting the predetermined electrical signal into the patient's organ;
(e) during the subsequent data collection session, registering a resulting electrical signal from the organ, the electrical signal configured as a second series of waveforms;
(f) generating from the second series of waveforms, a registered waveform representative of the average characteristics of the waveforms collected during the subsequent data collection session; and
(g) comparing the registered waveform to the reference waveform to determine whether the organ is functioning properly.

27. The method of claim 26 in which step (e) is carried out by:
(a) measuring the difference between at least one element of the registered waveform and a corresponding element of the reference waveform; and
(b) characterizing the difference in a scale of rejection in which a greater degree of difference corresponds to a greater degree of allograft rejection.

28. The method of claim 26 further comprising repeating steps (c)-(e) at selected intervals after the reference time so as to generate a plurality of registered waveforms.

29. The method of claim 26 in which step (e) is carried out by:
(a) adding the plurality of registered waveforms to a statistical database to create a data population;
(b) determining at least one difference between the registered waveforms and the reference waveform based on a statistical analysis of a plurality of elements of the registered waveforms and corresponding elements of the reference waveform; and
(c) characterizing the difference in a scale of rejection in which a greater degree of difference corresponds to a greater degree of allograft rejection.

30. The method of claim 26 further comprising:
(h) prior to step (b), evaluating whether each of the waveforms in the first series is usable according to a predetermined standard;
(i) discarding waveforms from the first series which are not usable; and
(j) storing the remaining waveforms of the first series in a database for use in generating the reference waveform;

31. The method of claim 26 further comprising:
(f) prior to step (b), evaluating whether each of the waveforms in the subsequent series is usable according to a predetermined standard;
(g) discarding waveforms from the subsequent series which are not usable; and
(h) storing the remaining waveforms of the subsequent series in a database for use in generating the registered waveform.

32. A method of monitoring a transplanted organ, comprising:
(a) during a data collection session, injecting a predetermined electrical signal into a patient's organ, the electrical signal configured as a series of waveforms;
(b) during the data collection session, registering a resulting electrical signal from a patient's organ, the electrical signal configured as a series of waveforms;
(c) evaluating whether each of the waveforms is usable according to a predetermined standard;
(d) discarding waveforms which are not usable;
(e) storing the remaining waveforms in a database for evaluation; and
(f) comparing the stored waveforms to a reference waveform to determine whether the organ is functioning properly.

33. The method of claim 32 further comprising:
(a) incrementing a discard counter each time a waveform is discarded;
(b) comparing the value of the discard counter to a predetermined limit; and
(c) setting an error flag if the discard counter exceeds a predetermined limit.

34. The method of claim 32 further comprising generating, from the remaining waveforms, an average waveform representative of the average characteristics of all of the waveforms collected during the data collection session.

35. The method of claim 32 further comprising:
(f) storing a first set of waveforms according to steps (a)-(e) during a first data session occurring at a reference time;
(g) generating from the remaining waveforms in the first data collection session, the reference waveform, wherein the reference waveform is representative of the average characteristics of the waveforms stored during the first data collection session;
(h) storing a subsequent set of waveforms according to steps (a)-(e) during a subsequent data collection session occurring at a time subsequent to the reference time; and
(i) generating from the remaining waveforms in the subsequent data collection session, a registered waveform representative of the average characteristics of the waveforms stored during the subsequent data collection session.

36. The method of claim 35 comprising repeating steps (h) and (i) at selected intervals after the reference time so as to generate a plurality of registered waveforms.

37. The method of claim 35 wherein the reference time is shortly after the organ is transplanted into the patient.

38. A method of processing data for monitoring a patient's organ, comprising:
(a) during a data collection session, injecting an electrical signal into a patient's organ;
(b) during the data collection session, registering a resulting electrical signal from the organ, the electrical signal configured as a series of waveforms, wherein each of said waveform includes at least one upslope element extending to a peak;
(c) establishing a minimum slope value;
(d) comparing the actual slope value of each portion of the upslope to the minimum slope value; and
(e) designating any point within the waveform in which the actual slope value is less than the minimum slope value to be a peak.

39. The method of claim 38 further comprising:
(a) establishing the time value at which the peak occurs;
(b) applying a hysteresis band with predetermined upper and lower voltage limits to the waveform;
(c) calculating a voltage-time slope for a segment of the upslope immediately preceding the peak;
(d) using the calculated voltage-time slope, linearly extrapolating the upslope to a point at which a voltage value thereof intercepts the upper limit of the hysteresis band; and
(e) establishing the time value at which the interception occurs.
